(19) United States
(12) Patent Application Publication
(10) Pub. No.: US 2026/0047835 A1
(43) Pub. Date: Feb. 19, 2026

(54) VASCULAR SEALING DEVICE

(71) Applicant: MYODYNAMICS, LLC, Englewood Cliffs, NJ (US)

(72) Inventors: Sreejit Nair, Pembroke Pines, FL (US); Ashwani Sastry, Orlando, FL (US)

(21) Appl. No.: 19/231,456

(22) Filed: Jun. 7, 2025

Related U.S. Application Data
(63) Continuation-in-part of application No. 18/805,278, filed on Aug. 14, 2024.

(51) Int. Cl. A61B 17/00; A61B 17/12; A61B 90/00

(52) U.S. Cl. CPC A61B 17/0057; A61B 2017/00557; A61B 2017/00592; A61B 2017/00623; A61B 2017/00659; A61B 2017/00672; A61B 2017/00778; A61B 2017/12004; A61B 2090/3966

(57) ABSTRACT

The present disclosure relates to a vascular sealing device. The vascular sealing device includes a guidewire, a sheath member, and an occlusion plug. The guidewire is deployable into a blood vessel through a vascular access point of a subject. The sheath member defines a hollow cavity along a length of the sheath member. The hollow cavity allows insertion of the guidewire therein. The sheath member is deployable from the guidewire and is configured to be positioned in a lumen of the blood vessel. The occlusion plug is deployable from the sheath member and positioned at the lumen of the blood vessel. The occlusion plug selectively operates in an inflated configuration and a deflated configuration to apply a controlled pressure to an interior portion of the blood vessel and an exterior portion of the blood vessel at the vascular access point to occlude the vascular access point of the blood vessel.

### TECHNICAL FIELD

The present disclosure relates to medical devices, and more particularly, to a vascular sealing device designed to control hemorrhage/hemostasis at a vascular access point during or post-surgical procedures.

### BACKGROUND

Endovascular surgery is a minimally invasive procedure used to treat various vascular conditions by accessing blood vessels through small incisions, typically in the groin or arm, rather than through large incisions required in traditional open surgery. This approach involves performing procedures inside blood vessels using specialized instruments such as catheters, stents, balloons, and embolic agents. The surgery is usually guided by real-time imaging techniques, primarily fluoroscopy, ultrasound, and sometimes computed tomography (CT) or magnetic resonance imaging (MRI), which provide the surgeon with live feedback of the vascular structures being treated.

After an endovascular procedure, manual compression of the arterial access site for hemostasis is a common technique used to stop bleeding. Bleeding can occur during endovascular procedures for various reasons, ranging from mild oozing to severe hemorrhage. To prevent or stop bleeding (hemostasis), trained physicians employ different techniques and interventions. Traditionally, hemostasis is achieved by manually applying pressure to the puncture site to compress the blood vessel and encourage clot formation at the entry point. However, this traditional method has several drawbacks, including prolonged compression times, patient and operator discomfort, inconsistent pressure application, and reduced efficiency in achieving successful hemostasis.

In recent years, Vascular Closure Devices (VCDs) have been used to stop bleeding and close arterial access sites following minimally invasive vascular procedures like cardiac catheterization, angiography, or percutaneous vascular interventions. However, using VCDs requires proper training and expertise to minimize complications such as bleeding, hematoma formation, pseudoaneurysms, or vascular injury. Therefore, selecting and deploying a VCD should be based on clinical judgment, patient characteristics, and the specifics of the procedure. Despite their use, current VCDs have certain limitations. They can be difficult to operate, requiring a steep learning curve and increasing the likelihood of user error. Additionally, even when used correctly, some VCDs may fail to achieve effective hemostasis or access site closure, leading to prolonged bleeding or inadequate sealing. Moreover, complications from using these devices may necessitate further interventions or surgery.

Therefore, there is a need for an efficient device to overcome one or more limitations stated above, in addition to providing other technical advantages.

### SUMMARY

Various embodiments of the present disclosure provide a vascular sealing device.

In an embodiment, a vascular sealing device is disclosed. The vascular sealing device includes a guidewire and a sheath member. The guidewire is adapted to be deployable into a blood vessel through a vascular access point of a subject. The sheath member defines a hollow cavity along a length of the sheath member. The hollow cavity is configured to allow insertion of the guidewire therein. The sheath member is deployable from the guidewire and is configured to be positioned in a lumen of the blood vessel. The vascular sealing device further includes at least one occlusion plug. The at least one occlusion plug is deployable from the sheath member and is configured to be positioned at the lumen of the blood vessel. The at least one occlusion plug is configured to selectively operate in an inflated configuration and a deflated configuration to apply a controlled pressure to an interior portion of the blood vessel and an exterior portion of the blood vessel at the vascular access point to occlude the vascular access point of the blood vessel.

In another embodiment, a vascular sealing device is disclosed. The vascular sealing device includes a guidewire and a sheath member. The guidewire is adapted to be deployable into a blood vessel through a vascular access point of a subject. The sheath member defines a hollow cavity along a length of the sheath member. The hollow cavity is configured to allow insertion of the guidewire therein. The sheath member is deployable from the guidewire and is configured to be positioned in a lumen of the blood vessel. The vascular sealing device further includes at least one occlusion plug. The at least one occlusion plug is deployable from the sheath member and is configured to be positioned at the lumen of the blood vessel. Further, the at least one occlusion plug includes a first occlusion plug and a second occlusion plug. The first occlusion plug includes a first shaft member and a first occluding member. The first occluding member is mounted to a distal end of the first shaft member. The first occlusion plug is deployable through the sheath member and is configured to be positioned within the lumen of the blood vessel. Further, the second occlusion plug includes a second shaft member and a second occluding member. The second occluding member is mounted to a distal end of the second shaft member. The second occlusion plug is deployable through the sheath member and is configured to be positioned at an exterior portion of the lumen of the blood vessel. The at least one occlusion plug is configured to selectively operate in an inflated configuration and a deflated configuration to apply a controlled pressure to an interior portion of the blood vessel and the exterior portion of the blood vessel at the vascular access point to occlude the vascular access point of the blood vessel.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or tool and instrumentalities disclosed herein. Moreover, those in the art will understand that the drawings are not to scale. Wherever possible, like elements have been indicated by identical numbers:

FIG. 1A illustrates a schematic representation of a subject undergoing an endovascular procedure, in accordance with an example embodiment of the present disclosure;

FIG. 1B illustrates a schematic view of a vascular sealing device being inserted into the subject, in accordance with an embodiment of the present disclosure;

FIG. 2 illustrates an exploded view of the vascular sealing device, in accordance with an embodiment of the present disclosure; and

FIGS. 3A-3H illustrates an example scenario depicting the vascular sealing device being used to achieve hemostasis, in accordance with an embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### Overview

In an embodiment, a vascular sealing device is disclosed. The vascular sealing device includes a guidewire and a sheath member. The guidewire is adapted to be deployable into a blood vessel through a vascular access point of a subject. The sheath member defines a hollow cavity along a length of the sheath member. The hollow cavity is configured to allow insertion of the guidewire therein. The sheath member is deployable from the guidewire and is configured to be positioned in a lumen of the blood vessel. The vascular sealing device further includes at least one occlusion plug. The at least one occlusion plug is deployable from the sheath member and is configured to be positioned at the lumen of the blood vessel. The at least one occlusion plug is configured to selectively operate in an inflated configuration and a deflated configuration to apply a controlled pressure to an interior portion of the blood vessel and an exterior portion of the blood vessel at the vascular access point to occlude the vascular access point of the blood vessel.

Further, the at least one occlusion plug includes a first occlusion plug and a second occlusion plug. The first occlusion plug includes a first shaft member and a first occluding member. The first occluding member is mounted to a distal end of the first shaft member. The first occlusion plug is deployable through the sheath member and is configured to be positioned within the lumen of the blood vessel. Further, the second occlusion plug includes a second shaft member and a second occluding member. The second occluding member is mounted to a distal end of the second shaft member. The second occlusion plug is deployable through the sheath member and is configured to be positioned at an exterior portion of the lumen of the blood vessel.

The first occluding member is deployed in the lumen of the blood vessel. The first occluding member is configured to be operated in an inflated configuration and a deflated configuration to apply the focal pressure on an interior portion of the blood vessel. The first occluding member conforms to the interior portion of the blood vessel while the first occlusion plug is operated in the inflated configuration, thereby occluding the blood vessel.

Further, the second occluding member is deployed at the exterior portion of the blood vessel. The second occluding member is configured to be operated in an inflated configuration and a deflated configuration to apply the focal pressure on the exterior portion of the blood vessel to occlude the vascular access point of the blood vessel. The second occlusion plug is configured to receive a hemostatic agent for operating the second occluding member in the inflated configuration to achieve hemostasis.

Furthermore, at least one of the first shaft member and the second shaft member is configured with measurement markers. The measurement markers guide the positioning of the second occlusion plug at the exterior portion of the lumen of the blood vessel. The at least one occlusion plug corresponds to a balloon catheter. The sheath member and the at least one occlusion plug are made of biocompatible materials. The at least one occlusion plug, the sheath member, and the guidewire are configured to incorporate radio-opaque contrast materials.

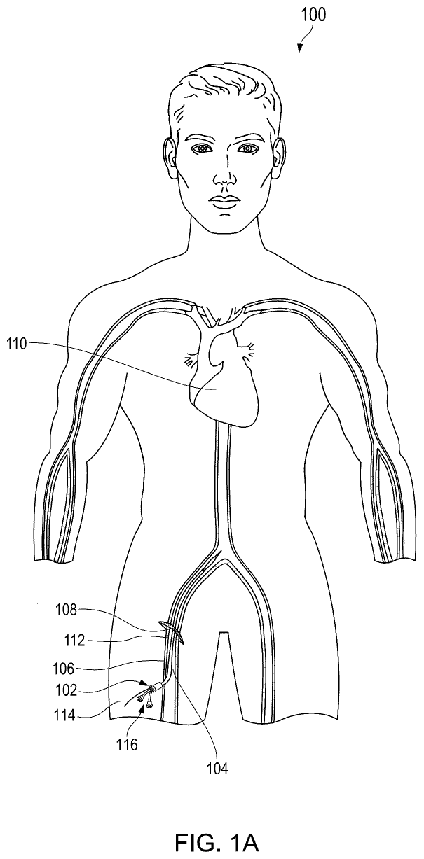

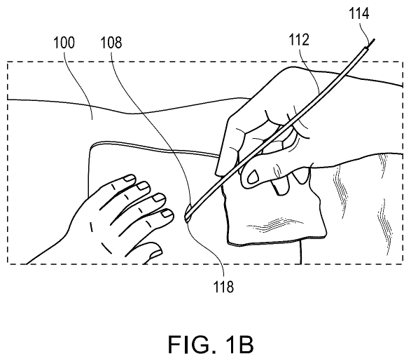

Various embodiments of the present disclosure are described with reference to FIGS. 1A-1B to FIGS. 3A-3H.

FIG. 1A illustrates a schematic representation of a subject 100 undergoing a medical procedure, in accordance with an example embodiment of the present disclosure. As shown, a vascular sealing device 102 is deployed into a lumen 104 of a blood vessel 106, through a vascular access point 108 of the subject 100. More specifically, the vascular sealing device 102 introduced into the lumen 104 of the blood vessel 106 provides access to an internal organ of the subject 100. For example, the vascular sealing device 102 may be deployed into a femoral artery. The femoral artery is a common access point for performing the medical procedure (e.g., an endovascular procedure such as cardiac catheterization). Further, the vascular sealing device 102 may be used in other obstetric procedures such as postpartum hemorrhage control, gastrointestinal applications (e.g., esophageal or gastric bleeding), and the like.

In the representative example, the vascular sealing device 102 is introduced into the lumen 104 of the blood vessel 106 through the vascular access point 108 of the subject 100, using a retrograde approach. The retrograde approach involves inserting the vascular sealing device 102 from a location on or within the body of the subject 100 where access is gained against the normal flow of blood, fluids, or physiological movement, often to reach a target site that is difficult to access via standard (antegrade) pathways. In particular, the retrograde approach refers to the approach of entering the intravascular device 102 into the lumen 104 of the blood vessel 106, from the vascular access point 108 located at a distal site thereof. This allows the vascular sealing device 102 to travel upstream against the normal direction of blood flow, which is crucial for accessing the internal organs of the subject 100 during various procedures, such as cardiac catheterization. By navigating the vascular sealing device 102 through the vascular system, clinicians can target specific areas, such as, but not limited to, the coronary arteries, to address conditions like arterial blockages or stenosis. Those skilled in the art would appreciate that the size and location of the vascular sealing device 102 may vary depending on the specific procedure. For instance, when performing the retrograde approach to treat the coronary vasculature of a heart 110, the vascular sealing device 102 is advanced from the femoral artery into the descending aorta, then into the coronary arteries. Additionally, the retrograde approach involves positioning of the vascular sealing device 102 at the target site through the rectum and the nasal cavity.

The vascular sealing device 102 is configured to achieve hemostasis at the vascular access point 108 following an interventional procedure as mentioned above. In one example, the vascular sealing device 102 may be used for sealing arterial or venous puncture sites resulting from catheterization, percutaneous interventions, or other endovascular procedures.

The vascular sealing device 102 includes a sheath member 112, a guidewire 114, and at least one occlusion plug 116. The sheath member 112 is a thin flexible tube adapted to be inserted into the blood vessel 106 to permit the introduction or withdrawal of fluids or to keep the vascular access point 108 open. Typically, the sheath member 112 is deployed at the target site (or an operating site) of the subject 100 using the guidewire 114. In particular, the sheath member 112 is configured for deployment into the blood vessel 106 over the guidewire 114 that is pre-positioned in the blood vessel 106. This technique facilitates safe and accurate introduction of interventional devices into the vascular system and reduces the risk of vessel trauma or misplacement.

As shown in FIG. 1B, the vascular sealing device 102 is being inserted into the blood vessel 106 of the subject 100. Typically, a small percutaneous puncture (see, 118 of FIG. 1B) may be made to access the blood vessel 106 for providing access to the vascular sealing device 102. Thereafter, the guidewire 114 may be introduced through the blood vessel 106 through the puncture 118. Further, other components of the vascular sealing device 102 such as the sheath member 112 and the at least one occlusion plug 116 may be introduced into the blood vessel 106 as per the procedure to achieve hemostasis, which will be explained further in detail.

The size and location of the vascular access point 108 used to access the blood vessel 106 may vary based on the procedures involved in treating the internal organ (e.g., the heart 110). In an example, when treating the coronary vasculature of the heart 110, the vascular access point 108 may be the right groin, with the vascular sealing device 102 passing into the right coronary artery (as shown in FIG. 1A). In another example, in the case of performing an angiography (i.e., medical imaging technique in the heart 110), the guidewire 114 and the sheath member 112 are inserted in the coronary artery and chambers of the heart 110.

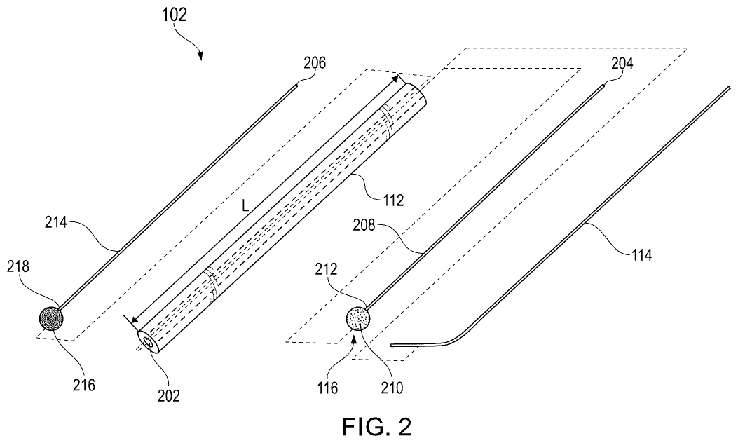

FIG. 2 illustrates an exploded view of the vascular sealing device 102, in accordance with an embodiment of the present disclosure. The vascular sealing device 102 includes the sheath member 112, the guidewire 114, and the at least one occlusion plug 116. The sheath member 112 may be configured to be an elongated tube. In other words, the sheath member 112 is configured to be a cylindrical structure. Alternatively, the sheath member 112 may be configured in any other structural configuration that serves the purpose. The sheath member 112 of the vascular sealing device 102 is an essential component designed to facilitate the delivery and deployment of the sealing elements, such as the anchor and at least one occlusion plug 116, within the vascular access point 108. The sheath member 112 is configured for percutaneous insertion into the blood vessel 106, providing a conduit (such as a hollow cavity 202) for the introduction of the sealing components as explained above. The hollow cavity 202 is defined along a length (L1) of the sheath member 112. As explained above, the sheath member 112 is deployable into the lumen 104 of the blood vessel 106 via the guidewire 114.

The guidewire 114 is a slender, flexible wire used as a navigational tool during percutaneous medical procedures. The guidewire 114 serves as a guiding conduit for the introduction of various interventional devices, such as the sheath member 112, into the blood vessel 106 as explained above. The guidewire 114 is designed to facilitate the precise positioning of the vascular sealing device 102 within the blood vessel 106 and ensure that the vascular sealing device 102 is deployed at the desired location (or the target site). The guidewire 114 is typically made from high-strength, biocompatible materials, such as stainless steel, nitinol, or a combination of metals and polymers. These materials provide the guidewire 114 with the necessary flexibility and durability to navigate through the blood vessel 106 while maintaining its structural integrity. The core of the guidewire 114 provides strength and rigidity, enabling it to be pushed and navigated through the blood vessel 106. In an embodiment, the guidewire 114 may be provided with an outer coating of hydrophilic material or Teflon, designed to reduce friction during insertion and facilitate smoother passage through the blood vessel 106. The guidewire 114 is often provided with radio-opaque contrast material (not shown in figures) to enable real-time visualization under fluoroscopy or X-ray imaging during the medical procedure.

Further, the sheath member 112 may be made from a flexible, biocompatible material, such as polyurethane, polyvinyl chloride (PVC), or a medical-grade thermoplastic elastomer, which allows for easy manipulation and insertion while maintaining structural integrity. In some embodiments, the sheath member 112 may be reinforced with braided or coiled metal wires to enhance its resistance to kinking and improve torque control during placement. It is to be noted that an outer circumference of the sheath member 112 is dimensioned to accommodate other components, such as the guidewire 114, the at least one occlusion plug 116, and other interventional tools. This configuration of the sheath member 112 is designed to accommodate standard guidewires and provide smooth passage of the guidewire 114 and the at least one occlusion plug 116 without causing friction or damage to the wall of the lumen 104 of the blood vessel 106. Thus, the sheath member 112 enables the controlled deployment of the at least one occlusion plug 116, typically in conjunction with the guidewire 114, which will be explained further in detail. After deployment, the sheath member 112 may be retracted or withdrawn, leaving the vascular sealing device 102 in place to close the puncture site. In an embodiment, the sheath member 112 may be configured with multiple hollow cavities to allow insertion of the guidewire 114 and the at least one occlusion plug 116, separately, without any interference between them. Additionally, the sheath member 112 may be incorporated with the radio-opaque contrast material (not shown in figures) to enable real-time visualization under fluoroscopy or X-ray imaging during the medical procedure.

The vascular sealing device 102 further includes the at least one occlusion plug 116. The at least one occlusion plug 116 is a medical device designed to block or seal off the blood vessel 106 or a body cavity of the subject 100 to prevent bleeding and promote hemostasis. In particular, the at least one occlusion plug 116 is deployable from the sheath member 112 and is configured to be positioned at the lumen 104 of the blood vessel 106. The at least one occlusion plug 116 is configured to selectively operate in an inflated configuration and a deflated configuration to apply a controlled pressure to an interior portion of the blood vessel 106 and an exterior portion of the blood vessel 106 at the vascular access point 108 to occlude the vascular access point 108 of the blood vessel 106. It is to be noted that the at least one occlusion plug 116 may be designed to conform to the anatomy of the blood vessel 106 or the puncture site (or the target site), exerting pressure against the wall of the blood vessel 106 to achieve a secure and reliable seal.

The at least one occlusion plug 116 includes a first occlusion plug 204 and a second occlusion plug 206. The first occlusion plug 204 includes a first shaft member 208 and a first occluding member 210 mounted to a distal end 212 of the first shaft member 208. The first occlusion plug 204 is deployable from the sheath member 112 and is configured to be positioned within the lumen 104 of the blood vessel 106. The second occlusion plug 206 includes a second shaft member 214 and a second occluding member 216 mounted to a distal end 218 of the second shaft member 214. The second occlusion plug 206 is deployable through the sheath member 112 and is configured to be positioned exterior to the lumen 104 of the blood vessel 106.

Further, the at least one occlusion plug 116 (including the first occlusion plug 204 and the second occlusion plug 206) is typically constructed from biocompatible, flexible materials such as collagen, polylactic acid (PLA), polyglycolic acid (PGA), polyethylene glycol (PEG), or polymers. In some embodiments, the components of the vascular sealing device 102 such as the sheath member 112 and the at least one occlusion plug 116 may be made of bioabsorbable materials. In this configuration, the at least one occlusion plug 116 and the sheath member 112 may be gradually decomposed and absorbed by the body of the subject 100 over a predetermined period. This allows for natural healing of the blood vessel 106 without the need for surgical removal of the at least one occlusion plug 116. Additionally, the at least one occlusion plug 116 (including the first occlusion plug 204 and the second occlusion plug 206) may be incorporated with the radio-opaque contrast material (not shown in figures) to enable real-time visualization under fluoroscopy or X-ray imaging during the medical procedure.

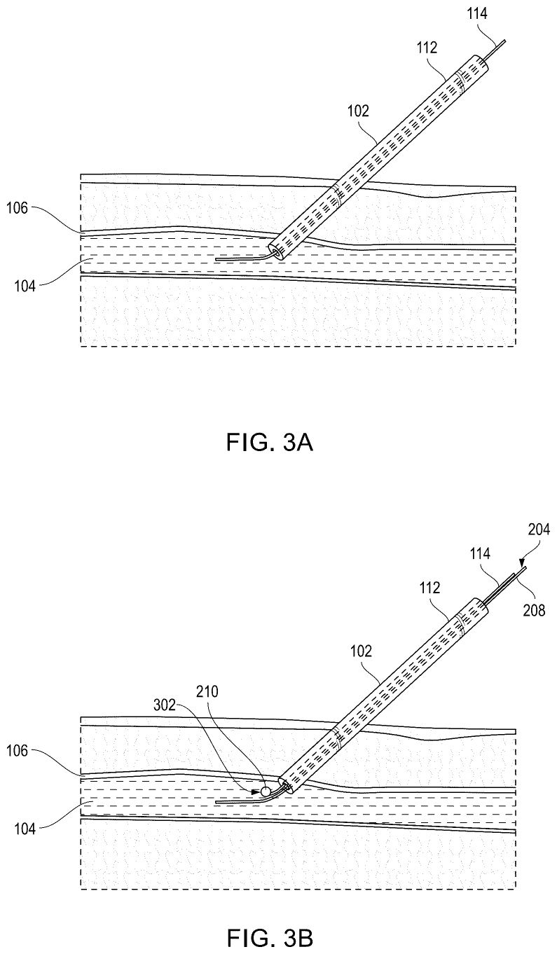

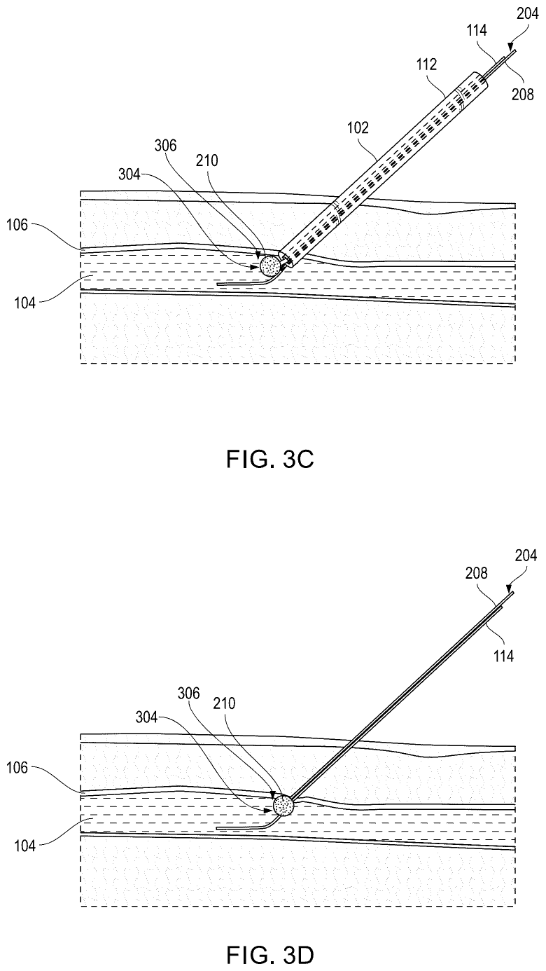

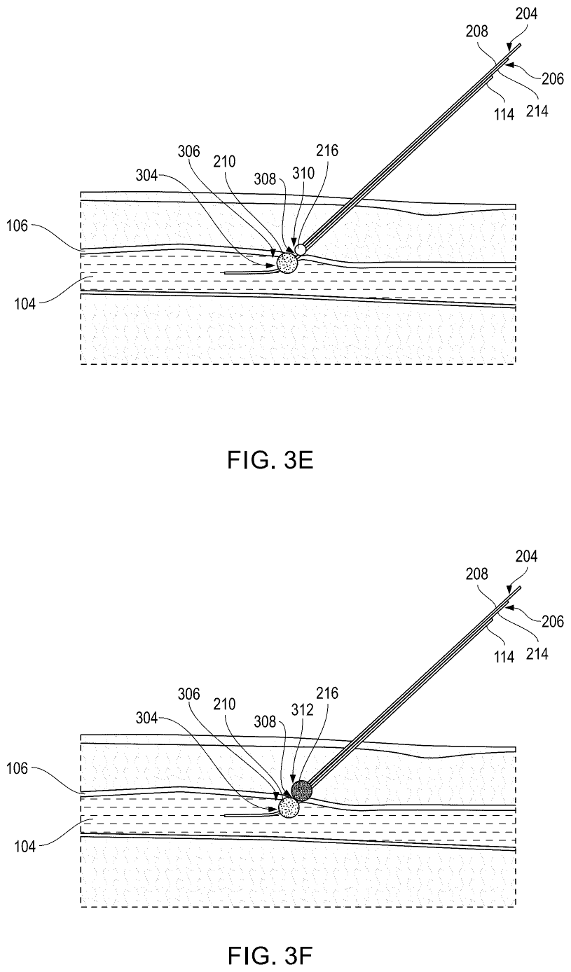

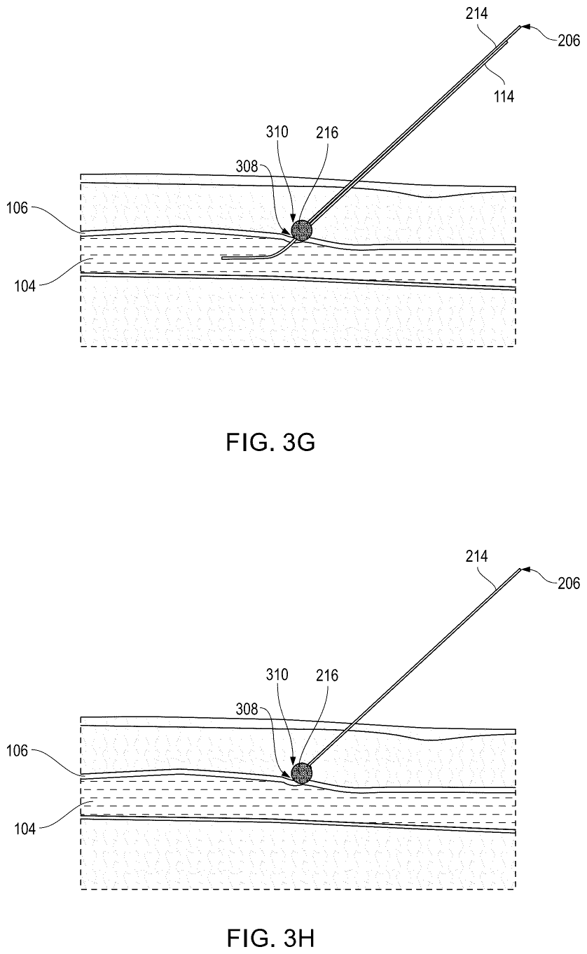

FIGS. 3A-3H illustrates an example scenario depicting the vascular sealing device 102 being used to achieve hemostasis, in accordance with an embodiment of the present disclosure. As explained above, the vascular sealing device 102 is used to achieve hemostasis at the vascular access point 108 following the interventional procedure (or the medical procedure) such as, but not limited to, catheterization, percutaneous interventions, or any other endovascular procedures.

Referring to FIG. 3A, the sheath member 112 and the guidewire 114 are introduced into the lumen 104 of the blood vessel 106. The sheath member 112 may be initially inserted into the blood vessel 106 during the diagnostic or the interventional procedure. Further, the guidewire 114 may be threaded through the sheath member 112 into the blood vessel 106 through the vascular access point 108. The guidewire 114 stabilizes device delivery, allowing for smooth and controlled advancement of the at least one occlusion plug 116 through tissue planes of the blood vessel 106. Further, the sheath member 112 serves as the primary access conduit through which the at least one occlusion plug 116 is delivered to the target site in the blood vessel 106. It must be noted that the sheath member 112 helps protect surrounding tissues during the introduction and manipulation of the at least one occlusion plug 116.

Referring to FIG. 3B, the first occlusion plug 204 is typically the first sealing element deployed into the blood vessel 106 (e.g., arteriotomy) from the sheath member 112 accommodated with the guidewire 114 through the vascular access point 108. Typically, the first occlusion plug 204 is advanced until the first occluding member 210 is positioned inside the lumen 104 of the blood vessel 106 (as shown in FIG. 3B). The first shaft member 208 of the first occlusion plug 204 may be configured to guide and align the first occlusion plug 204 over the guidewire 114 (in an axial manner). The first occluding member 210 may include a collapsible balloon configured at the distal end 212 of the first shaft member 208. Hence, the first occlusion plug 204 corresponds to a balloon catheter. For example, the first occlusion plug 204 may be made of elastomeric, biocompatible materials such as polyurethane or nylon.

Referring to FIG. 3C, the first occluding member 210 being deployed in the lumen 104 of the blood vessel 106 is operated in an inflated configuration 304 from a deflated configuration (see, 302 of FIG. 3B) to apply the focal pressure on an interior portion 306 of the blood vessel 106. It is to be noted that the first occluding member 210 conforms to the interior portion 306 of the blood vessel 106 while the first occlusion plug 204 is operated in the inflated configuration 304. This results in occluding the blood vessel 106. In other words, the first occluding member 210 once operated in the inflated configuration 304, the first occluding member 210 seals the blood vessel 106 inside temporarily and acts as a positional anchor. The first occluding member 210 may be operated in the inflated configuration 304 and the deflated configuration 302 using a connected syringe or an inflation handle, introducing a measured amount of fluid (typically saline, contrast media, or air), and the like. Further, the sheath member 112 is withdrawn from the blood vessel 106 (as shown in FIG. 3D).

Referring to FIG. 3E, the second occlusion plug 206 is deployed at the blood vessel 106 (typically above the blood vessel 106, such as the arteriotomy), once the first occlusion plug 204 is positioned in the lumen 104 of the blood vessel 106. Typically, the second occlusion plug 206 is advanced until the second occluding member 216 is positioned at an exterior portion (see, 308 of FIG. 3E) to the lumen 104 of the blood vessel 106 (as shown in FIG. 3F). The second shaft member 214 of the second occlusion plug 206 may be configured to guide and align the second occlusion plug 206 over the guidewire 114 (in an axial manner). Additionally, at least one of the first shaft member 208 and the second shaft member 214 may be configured with measurement markers (not shown in figures). The measurement markers guide the positioning of the second occlusion plug 206 at the exterior portion 308 of the lumen 104 of the blood vessel 106. Herein, the exterior portion 308 of the lumen 104 of the blood vessel 106 corresponds to a portion between the vessel wall and the skin tissue layer. For example, the measurement markers may include radiopaque markers to confirm correct positioning under fluoroscopy or ultrasound.

Further, the second occluding member 216 may include a collapsible balloon configured at the distal end 218 of the second shaft member 214. Hence, the second occlusion plug 206 corresponds to a balloon catheter. For example, the second occlusion plug 206 may be made of elastomeric, biocompatible materials such as polyurethane or nylon.

Referring to FIG. 3F, the second occluding member 216 is deployed at the exterior portion 308 of the blood vessel 106. The second occluding member 216 is configured to be operated between an inflated configuration (see, 312 of FIG. 3F) and a deflated configuration (see, 310 of FIG. 3E) to apply the focal pressure on the exterior portion 308 of the blood vessel 106 to occlude the vascular access point 108 of the blood vessel 106. In other words, the second occluding member 216 applies counter-pressure from above, forming a plug sandwich with the vessel wall (i.e., the exterior portion 308) there between.

Referring to FIG. 3G, the first occluding member 210 may be operated in the deflated configuration 302, and thereafter, the first occlusion plug 204 may be gently removed from the blood vessel 106, keeping the second occlusion plug 206 and the guidewire 114 in place. The guidewire 114 is positioned in the blood vessel 106 for allowing the sheath member 112 to be positioned again in the lumen 104 of the blood vessel 106 in case the hemostasis fails.

Referring to FIG. 3H, once hemostasis has been achieved at the target site in the blood vessel 106, the guidewire 114 may be removed from the blood vessel 106. Further, the second occlusion plug 206 may be held at the target site in the blood vessel 106 for a predefined time to achieve hemostasis. Once the hemostasis is achieved, the second occlusion plug 206 is operated in the deflated configuration 310. Thereafter, the second occlusion plug 206 is gently removed from the blood vessel 106. Additionally, the second occlusion plug 206 (specifically, the second occluding member 216) may be configured to receive a hemostatic agent for operating the second occluding member 216 in the inflated configuration 312 to achieve hemostasis. The hemostatic agent typically expedites/accelerates the hemostasis process. Some examples of the hemostatic agents include collagen, gelatin, fibrin, chitosan, and the like.

The vascular sealing device 102 described herein provides a highly effective system for achieving hemostasis following percutaneous vascular access procedures. Further, the dual-plug system (such as the first occlusion plug 204 and the second occlusion plug 206) provides a mechanical and hemostatic barrier from both the inside and outside of the blood vessel 106, resulting in a more reliable seal compared to other traditional closure systems (e.g., single-plug or suture-based closure systems) . Moreover, the combination of the first occlusion plug 204 and the second occlusion plug 206 exerts internal and external pressure, respectively, to seal the blood vessel 106, thus significantly reducing the time required to achieve hemostasis. This is particularly beneficial in high-flow vessels such as the femoral or radial arteries, where prolonged bleeding could lead to complications such as hematoma, pseudoaneurysm, or prolonged hospitalization. The vascular sealing device 102 is engineered for cost-effective manufacturing and clinical use. Additionally, the vascular sealing device 102 is designed to be deployed using existing vascular access tools (e.g., guidewires, sheaths, and introducers). This configuration of the vascular sealing device 102 reduces the need for specialized or proprietary equipment, thus resulting in eliminating the additional procedural steps, minimizing the need for expertise training. Hence, the vascular sealing device 102 is a cost-efficient device for achieving the hemostasis.

Various embodiments of the disclosure, as discussed above, may be practiced with steps and/or operations in a different order, and/or with hardware elements in configurations, which are different than those which, are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A vascular sealing device, comprising:
a guidewire adapted to be deployable into a blood vessel through a vascular access point of a subject;
a sheath member defining a hollow cavity along a length of the sheath member, the hollow cavity configured to allow insertion of the guidewire therein, wherein the sheath member is deployable from the guidewire and configured to be positioned in a lumen of the blood vessel; and
at least one occlusion plug deployable from the sheath member and configured to be positioned at the lumen of the blood vessel, wherein the at least one occlusion plug is configured to selectively operate in an inflated configuration and a deflated configuration to apply a controlled pressure to an interior portion of the blood vessel and an exterior portion of the blood vessel at the vascular access point to occlude the vascular access point of the blood vessel.

2. The vascular sealing device as claimed in claim 1, wherein the at least one occlusion plug comprises:
a first occlusion plug comprising a first shaft member and a first occluding member mounted to a distal end of the first shaft member, wherein the first occlusion plug is deployable through the sheath member and configured to be positioned within the lumen of the blood vessel; and
a second occlusion plug comprising a second shaft member and a second occluding member mounted to a distal end of the second shaft member, wherein the second occlusion plug is deployable through the sheath member and configured to be positioned at an exterior portion of the lumen of the blood vessel.

3. The vascular sealing device as claimed in claim 2, wherein the first occluding member is deployed in the lumen of the blood vessel, the first occluding member configured to be operated in an inflated configuration and a deflated configuration to apply the focal pressure on an interior portion of the blood vessel, and wherein the first occluding member conforms to the interior portion of the blood vessel while the first occlusion plug is operated in the inflated configuration, thereby occluding the blood vessel.

4. The vascular sealing device as claimed in claim 2, wherein the second occluding member is deployed at the exterior portion of the blood vessel, the second occluding member configured to be operated in an inflated configuration and a deflated configuration to apply the focal pressure on the exterior portion of the blood vessel to occlude the vascular access point of the blood vessel.

5. The vascular sealing device as claimed in claim 4, wherein the second occlusion plug is configured to receive a hemostatic agent for operating the second occluding member in the inflated configuration to achieve hemostasis.

6. The vascular sealing device as claimed in claim 2, wherein at least one of the first shaft member and the second shaft member is configured with measurement markers, the measurement markers guide the positioning of the second occlusion plug at the exterior portion of the lumen of the blood vessel.

7. The vascular sealing device as claimed in claim 1, wherein the at least one occlusion plug corresponds to a balloon catheter.

8. The vascular sealing device as claimed in claim 1, wherein the sheath member and the at least one occlusion plug are made of biocompatible materials.

9. The vascular sealing device as claimed in claim 1, wherein the at least one occlusion plug, the sheath member, and the guidewire are configured to incorporate radio-opaque contrast materials.

10. A vascular sealing device, comprising:
a guidewire adapted to be deployable into a blood vessel through a vascular access point of a subject;
a sheath member defining a hollow cavity along a length of the sheath member, the hollow cavity configured to allow insertion of the guidewire therein, wherein the sheath member is deployable from the guidewire and configured to be positioned in a lumen of the blood vessel; and
at least one occlusion plug deployable from the sheath member and configured to be positioned at the lumen of the blood vessel, the at least one occlusion plug comprising:
a first occlusion plug comprising a first shaft member and a first occluding member mounted to a distal end of the first shaft member, wherein the first occlusion plug is deployable through the sheath member and configured to be positioned within the lumen of the blood vessel, and
a second occlusion plug comprising a second shaft member and a second occluding member mounted to a distal end of the second shaft member, wherein the second occlusion plug is deployable through the sheath member and configured to be positioned at an exterior portion of the lumen of the blood vessel,
wherein the at least one occlusion plug is configured to selectively operate in an inflated configuration and a deflated configuration to apply a controlled pressure to an interior portion of the blood vessel and the exterior portion of the blood vessel at the vascular access point to occlude the vascular access point of the blood vessel.

11. The vascular sealing device as claimed in claim 10, wherein the first occluding member is deployed in the lumen of the blood vessel, the first occluding member configured to be operated in the inflated configuration and the deflated configuration to apply the focal pressure on the interior portion of the blood vessel, and wherein the first occluding member conforms to the interior portion of the blood vessel while the first occlusion plug is operated in the inflated configuration, thereby occluding the blood vessel.

12. The vascular sealing device as claimed in claim 10, wherein the second occluding member is deployed at the exterior portion of the blood vessel, the second occluding member configured to be operated in the inflated configuration and the deflated configuration to apply the focal pressure on the exterior portion of the blood vessel to occlude the vascular access point of the blood vessel.

13. The vascular sealing device as claimed in claim 12, wherein the second occlusion plug is configured to receive a hemostatic agent for operating the second occluding member in the inflated configuration to achieve hemostasis.

14. The vascular sealing device as claimed in claim 10, wherein at least one of the first shaft member and the second shaft member is configured with measurement markers, the measurement markers guide the positioning of the second occlusion plug at the exterior portion of the lumen of the blood vessel.

15. The vascular sealing device as claimed in claim 10, wherein the at least one occlusion plug corresponds to a balloon catheter.

16. The vascular sealing device as claimed in claim 10, wherein the sheath member and the at least one occlusion plug are made of biocompatible materials.

17. The vascular sealing device as claimed in claim 10, wherein the at least one occlusion plug, the sheath member, and the guidewire are configured to incorporate radio-opaque contrast materials.
